(19) United States
(12) Patent Application Publication
(10) Pub. No.: US 2002/0010221 A1
(43) Pub. Date: Jan. 24, 2002

(54) Fischer-tropsch processes and catalysts with promoters

(76) Inventors: Olga Ionkina, Ponca City, OK (US); Munirpallam A. Subramanian, Kennett Sq., PA (US); Wenchun Chao, State College, PA (US); Kamel M. Makar, Wilmington, DE (US); Leo E. Manzer, Wilmington, DE (US)

Correspondence Address: Joanna K. Payne, CONOCO INC., 1000 South Pine 2635 RW, P O Box 1267, Ponca City, OK, 74602-1267, US

(21) Appl. No.: 09/804,271

(22) Filed: Mar. 12, 2001

Related U.S. Application Data
(63) Continuation-in-part of application No. 09/314,811, filed on May 19, 1999.

(51) Int. Cl. B01J 23/34; B01J 23/58; C07C 1/04

(52) U.S. Cl. 518/715; 502/332; 502/324; 502/330; 502/207; 502/213

(57) ABSTRACT

A catalyst useful for the production of hydrocarbons from synthesis gas, in the Fischer-Tropsch reaction, is disclosed. The catalyst includes cobalt and rhenium and may be supported on an alumina support. The catalyst further includes a promoter selected from among boron, manganese, vanandium, phosphorous, and the alkali metals. The promoter preferably improves the activity for the production of hydrocarbons by at least about 5%. The improved activity may be an increased productivity, increased CO conversion, and the like. The hydrocarbons may have a weight range useful for making diesel fuel, (e.g. C₁₁⁺ hydrocarbons). Alternately, the hydrocarbons may have a weight range useful for making gasoline (e.g. C₅₊ hydrocarbons). The catalyst may be made by co-dispersing the promoter with the cobalt or by layering the promoter over the cobalt.

### RELATED APPLICATIONS

This application is a continuation-in-part application of U.S. patent application Ser. No. 09/414,811, filed May 19, 1999, hereby incorporated herein by reference in its entirety.

### STATEMENT REGARDING FEDERALLY SPONSORED RESEARCH OR DEVELOPMENT

Not Applicable.

### TECHNICAL FIELD OF THE INVENTION

The present invention relates to a catalyst and process for the preparation of hydrocarbons from synthesis gas, i.e., a mixture of carbon monoxide and hydrogen, typically labeled the Fischer-Tropsch process. More particularly, this invention relates to catalysts containing cobalt and rhenium and at least one other element selected from boron, phosphorous, vanadium, and manganese and the use of these catalysts in the Fischer-Tropsch process.

### BACKGROUND

Large quantities of methane, the main component of natural gas, are available in many areas of the world, and natural gas reserves are predicted to outlast oil reserves by a significant margin. However, most natural gas is situated in areas that are geographically remote from population and industrial centers. The costs of compression, transportation, and storage make its use economically unattractive. To improve the economics of natural gas use, much research has focused on the use of methane as a starting material for the production of higher hydrocarbons and hydrocarbon liquids, which are more easily transported and thus more economical. The conversion of methane to hydrocarbons is typically carried out in two steps. In the first step, methane is converted into a mixture of carbon monoxide and hydrogen (i.e., synthesis gas or syngas). In a second step, the syngas is converted into hydrocarbons.

This second step, the preparation of hydrocarbons from synthesis gas, is well known in the art and is usually referred to as Fischer-Tropsch synthesis, the Fischer-Tropsch process, or Fischer-Tropsch reaction(s). Fischer-Tropsch synthesis generally entails contacting a stream of synthesis gas with a catalyst under temperature and pressure conditions that allow the synthesis gas to react and form hydrocarbons.

More specifically, the Fischer-Tropsch reaction is the catalytic hydrogenation of carbon monoxide to produce any of a variety of products ranging from methane to higher alkanes and aliphatic alcohols. Research continues on the development of more efficient Fischer-Tropsch catalyst systems and reaction systems that increase the selectivity for high-value hydrocarbons in the Fischer-Tropsch product stream.

In particular, Fischer-Tropsch product mixtures containing, for example, C₅₊ hydrocarbons are useful for further processing to yield gasoline, whereas Fischer-Tropsch product mixtures containing, for example, C₁₁₊ hydrocarbons are useful for further processing to yield diesel fuel. For example, see the article “Short history and present trends of Fischer-Tropsch synthesis,” by H. Schlutz in Applied Catalysis A, vol. 186, pp. 3-12 (1999), hereby incorporated herein by reference in its entirety.

There are continuing efforts to find catalysts that are more effective at producing these desired products. Product distribution, product selectivity, and reactor productivity depend heavily on the type and structure of the catalyst and on the reactor type and operating conditions. It is particularly desirable to maximize the production of high-value liquid hydrocarbons, such as hydrocarbons with five or more carbon atoms per hydrocarbon chain (C₅₊).

Catalysts conventionally include a support material. Catalyst supports for catalysts used in Fischer-Tropsch synthesis of hydrocarbons have typically been oxides (e.g., silica, alumina, titania, zirconia or mixtures thereof, such as silica-alumina). The products prepared using these catalysts usually have a very wide range of molecular weights. It has been asserted that the Fischer-Tropsch synthesis reaction is only weakly dependent on the chemical identity of the metal oxide support. For example, see E. Iglesia et al. 1993, In: “Computer-Aided Design of Catalysts,” ed. E. R. Becker et al., p. 215, New York, Marcel Dekker, Inc., hereby incorporated herein by reference in its entirety.

Catalysts for use in the Fischer-Tropsch synthesis usually contain a catalytically active metal of Groups 8, 9, 10 (in the New notation of the periodic table of the elements, which is followed throughout). In particular, iron, cobalt, nickel, and ruthenium have been abundantly used as the catalytically active metals. Nickel is useful for a process in which methane is a desired product. Iron has the advantage of being readily available. Ruthenium has the advantage of high activity but is relatively expensive and thus is typically used a promoter for another of the catalytic materials. Cobalt has the advantages of being more active than iron and more available than ruthenium and less selective to methane than nickel.

Thus, cobalt has been investigated as a catalyst for the production of hydrocarbons with weights corresponding to the range of the gasoline, diesel, and higher weight fractions of crude oil. In particular, cobalt has been found to be most suitable for catalyzing a process in which synthesis gas is converted to primarily hydrocarbons having five or more carbon atoms (i.e., where the C₅₊ selectivity of the catalyst is high).

Additionally, the catalysts often contain one or more promoters. Rhenium is a widely tested promoter for cobalt-containing Fischer-Tropsch catalysts. For example, International Publication Nos. WO 98/47618 and WO 98/47620, hereby incorporated herein by reference in their entirety, disclose the use of rhenium promoters and describe several functions served by the rhenium. However, disadvantages of the use of rhenium are its high cost and limited availability.

Notwithstanding the above teachings, it continues to be desirable to improve the activity of Fischer-Tropsch catalysts. In particular, promoters are desirable which will increase availability and reduce the cost of effective catalysts.

### SUMMARY OF THE INVENTION

The present invention features a catalyst for the Fischer-Tropsch reaction that contains cobalt and rhenium and an additional promoter that improves the activity of the catalyst.

One embodiment of the present invention features a Fischer-Tropsch catalyst that includes alumina, cobalt, and rhenium, and a promoter selected from among boron, phosphorous, vanadium, manganese, and an alkali metal that improves the activity for production of hydrocarbons from synthesis gas, preferably by at least 5%. The improved activity may be an increased productivity. Alternatively, the improved activity may be an increased CO conversion. The hydrocarbons may have a weight range suitable for processing to diesel fuel. In particular, the hydrocarbons may include C₁₁₊ hydrocarbons. Alternately, the hydrocarbons may have a weight range suitable for processing to gasoline. In particular, the hydrocarbons may include C₅₊ hydrocarbons.

Another embodiment of the present invention features a Fischer-Tropsch process that includes contacting a feed stream including hydrogen and carbon monoxide with a catalyst in a reaction zone maintained at conversion-promoting conditions effective to produce an effluent stream that includes hydrocarbons. The catalyst includes alumina, cobalt, rhenium, and a promoter selected from among boron, manganese, phosphorous, vanadium, and an alkali metal. Further, hydrocarbons are produced with an improved activity as compared to a similar catalyst without the promoter, preferably improved by at least 5%. The improved activity may be an increased productivity. Alternatively, the improved activity may be an increased CO conversion. The hydrocarbons may have a weight range suitable for processing to diesel fuel. In particular, the hydrocarbons may include C₁₁₊ hydrocarbons. Alternately, the hydrocarbons may have a weight range suitable for processing to gasoline. In particular, the hydrocarbons may include C₅₊ hydrocarbons.

Still another embodiment of the present invention features a Fischer-Tropsch process using a cobalt/rhenium catalyst made by a method that includes adding a promoter to the catalyst, where the promoter may be boron, phosphorous, vanadium, manganese, or an alkali metal in an amount effective to improve the activity of the catalyst for the Fischer-Tropsch reaction by at least about 5%. The promoter may be co-dispersed on a support with the cobalt. Alternatively, the promoter may be layered over the cobalt.

An advantage of a cobalt/rhenium catalyst including a promoter according to the preferred embodiments of the present invention is that a lesser amount of rhenium may be used to achieve the same catalytic activity as a similar corresponding cobalt/rhenium catalyst essentially without the promoter. In particular, the weight percent of rhenium in the catalysts may be reduced by a factor of up to at least 10, such as from 1% to 0.1%, without disadvantage to overall catalytic activity.

Thus, the present invention comprises a combination of features and advantages that enable it to overcome various problems of prior catalysts and processes. The various characteristics described above, as well as other features, will be readily apparent to those skilled in the art upon reading the following detailed description of the preferred embodiments of the invention.

It will be understood that as contemplated herein, an improved activity, such as an increase in productivity, e.g. the C₅₊ productivity or the C₁₁₊ productivity, or an increase in CO conversion, of a catalyst containing a promoter according to the preferred embodiments of the present invention may be calculated according to conventional methods. In particular, an improvement in activity may be given as a percent and computed as the ratio of the activity difference to the activity of the reference catalyst. The activity difference is between the activity of the promoted catalyst and the reference catalyst, where the reference catalyst is a similar corresponding catalyst having the nominally same amounts, e.g. by weight percent, of rhenium and cobalt. It will further be understood that as contemplated herein, an increase in productivity may be measured in any conventional units, e.g. gm product per hour per liter reactor volume or gm product per hour per kg catalyst.

### BRIEF DESCRIPTION OF THE DRAWINGS

For a detailed description of the preferred embodiment of the present invention, reference will now be made to the accompanying drawings, wherein:

FIG. 1 is a plot of productivity increase vs. boron content for boron-promoted catalysts as compared to a similar catalyst without boron;

FIG. 2 is a plot of productivity increase vs. manganese content for manganese-promoted catalysts as compared to a similar catalyst without manganese;

FIG. 3 is a plot of productivity increase vs. phosphorous content for phosphorous-promoted catalysts as compared to a similar catalyst without phosphorous;

FIG. 4 is a plot of productivity increase vs. vanadium content for vanadium-promoted catalysts as compared to a similar catalyst without vanadium;

FIG. 5 is a plot of productivity increase vs. manganese for manganese-promoted catalysts made with a first alumina support (Al-1) and an alternative alumina support (Al-2) as compared to similar catalysts without manganese and supported on Al-1 and Al-2, respectively;

FIG. 6 is a plot of productivity increase vs. rhenium content for manganese-promoted, boron-promoted, and phosphorous-promoted catalysts as compared to similar respective catalysts without manganese, boron, and phosphorous, respectively;

FIG. 7 is a plot of productivity for promoted catalysts having varying rhenium content on the first alumina support (Al-1); and

FIG. 8 is a plot of productivity for promoted catalysts having varying rhenium content on the alternative alumina support (Al-2).

### DETAILED DESCRIPTION OF THE PREFERRED EMBODIMENTS

The present catalyst contains a catalytically effective amount of cobalt and rhenium. The amount of cobalt and rhenium present in the catalyst may vary widely. Typically, the catalyst comprises from about 1 to 50% by weight (as the metal) of the total supported cobalt and rhenium per total weight of catalytic metal and support, preferably from about 1 to 30% by weight, and more preferably from about 1 to 25% by weight. Rhenium is added to the support in a concentration sufficient to provide a weight ratio of elemental rhenium:elemental cobalt of from about 0.001:1 to about 0.25:1, preferably from about 0.001:1 to about 0.05:1 (dry basis).

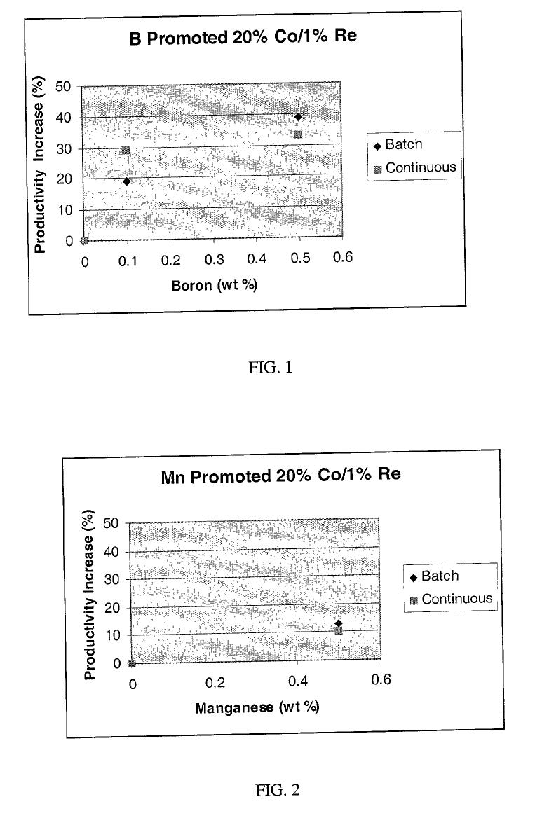

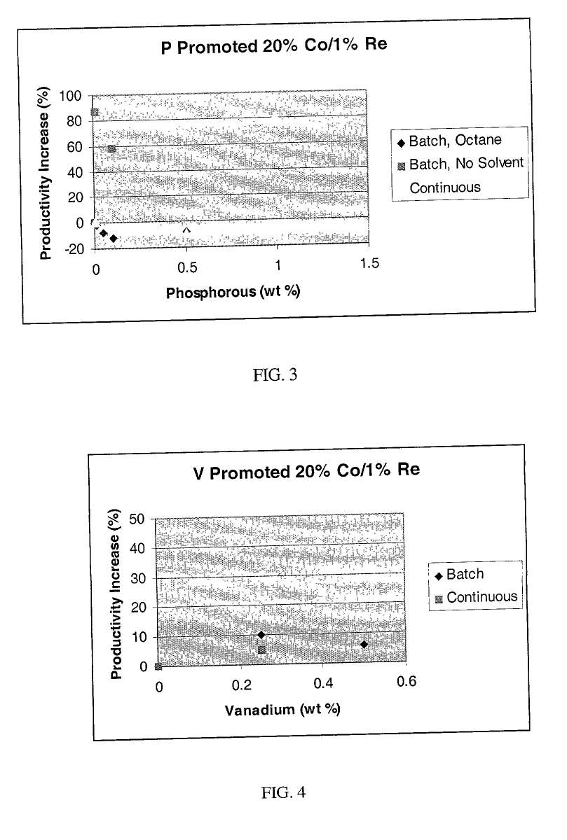

We have discovered that higher selectivity and productivity catalysts are produced when a promoter selected from the group consisting of boron, phosphorus, potassium, manganese and vanadium is added to the cobalt-rhenium catalyst. This is quite surprising because boron and phosphorus are typically considered to be Fischer-Tropsch catalyst poisons. Referring to FIGS. 1-6, results for the productivity increase obtained using the data presented below in Tables 1-7 and 13-14 are shown. In computing the productivity increase from the continuous data, results after 160 hours of operation were used, and the value of the reference productivity was 210 g/hr/kgcat, the average of the values for Examples 56-69. The reference catalysts used in computing the batch testing productivity increases were the respective controls from the same series of Examples, typically Example nA, where n is the Example number. Productivity increases having a magnitude of 4% are statistically significant, that is more than one standard deviation in the data. Similar results were obtained from batch and continuous testing for C₁₁₊ and C₅₊ productivities, respectively.

It will be understood that for a catalytic Fischer-Tropsch process producing hydrocarbons with a distribution of hydrocarbon weights described by a single Anderson-Shlutz-Flory chain growth probability α, values for the productivity increase will accordingly also be independent of carbon chain length. Typically, a single α will apply to liquid or wax hydrocarbons having at least 5 carbons. The Anderson-Shultz-Flory chain growth probability is well-known in the art and is described, for example, in Principles and Practices of Heterogeneous Catalysis, by J. M. thomas and W. J. Thomas (Verlagsgesellschaft mbH, New York, 1997), pp. 524-532, hereby incorporated herein by reference.

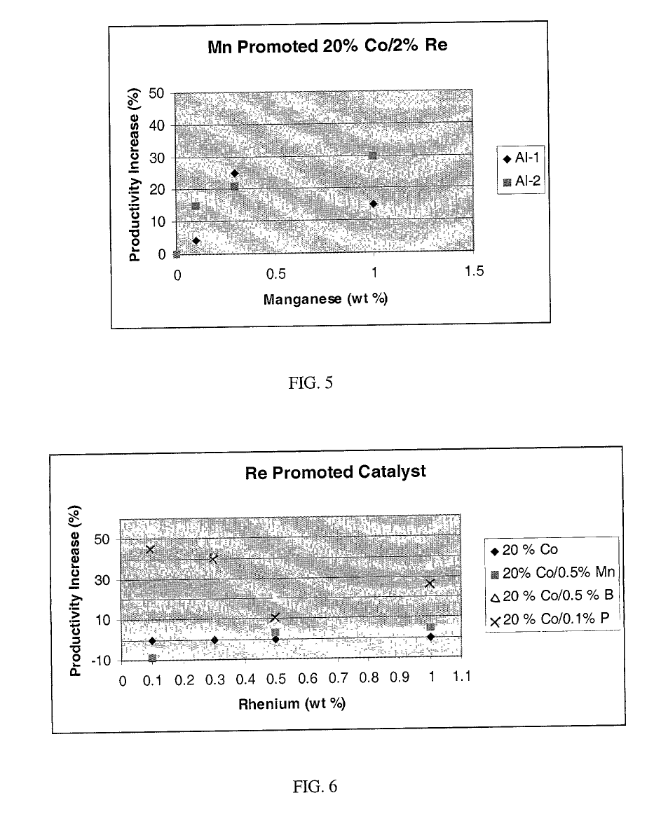

FIGS. 1-6 illustrate productivity increases of up to 5%, up to 10%, up to 20%, up to 30% and more, using promoted cobalt/rhenium catalysts. FIG. 6 further illustrates that the weight percent of rhenium in a rhenium-containing catalyst may be reduced by a factor of up to at least 10, such as from 1% to 0.1%, without disadvantage to overall catalytic activity by the addition of a promoter selected from boron, manganese, phosphorous, and vanadium.

Additionally, and even more surprisingly, the catalysts of the present invention exhibit both improved conversion and improved stability, with long lifetime, relative to prior art Fischer-Tropsch catalysts. The conversion is particularly high in the case of the boron and manganese promoted catalysts of the present invention, and can equal or approach 100%. Each of the preferred promoters is capable of increasing the C₁₁₊ productivity by at least 5%. Further, the increase in C₁₁₊ productivity is particularly high in the case of manganese and can exceed 45%. The C₁₁₊ productivity of the promoted catalyst is preferably increased by at least 5% as compared to a corresponding reference catalyst, still more preferably by at least 10%, and most preferably by at least about 20%. Further, each of the preferred promoters is capable of increasing the C₅₊ productivity by at least 5% as compared to a corresponding reference catalyst. The increase in C₅₊ productivity is particularly high in the case of manganese and can exceed 65%.

Surprisingly, the productivity increase relative to the reference unpromoted catalysts may rise with time, particularly in the case of the boron and manganese promoted catalysts. This effect may be attributed to the greater lifetime of these promoted catalysts as compared to similar unpromoted reference catalysts. Still further, a promoter selected from the group consisting of boron, manganese, phosphorous, and vanadium is capable of yielding the same productivity, such as C₁₁₊ productivity or C₅₊ productivity for a given catalyst as a corresponding catalyst having a lesser amount of rhenium.

Hence the amount of rhenium needed to achieve a given productivity is reduced, resulting in a cost savings. The amount of promoter is added to the cobalt-rhenium catalyst in a concentration sufficient to provide a weight ratio of elemental promoter:elemental cobalt of from about 0.00005:1 to about 0.5:1, preferably, from about 0.0005:1 to about 0.01:1 (dry basis). Further, the amount of promoter is preferably added to the cobalt-rhenium catalyst in a concentration sufficient to provide a weight ratio of elemental promoter:elemental rhenium of from about 0.01:1 to about 10:1 (dry basis).

The active catalyst components used in this invention may be carried or supported on any conventional support in any conventional form. These may include, but are not limited to any of the conventional alumina supports. We have found that the promoter activity of each promoter according to the preferred embodiments of the present invention is affected by the identity of the support. In particular, results differ for apparently similar alumina supports from different suppliers. We believe that a difference in dispersion of catalytic materials and promoter materials on the support may account for the observed differences in C₁₁₊ productivity between catalysts having alumina support material supplied by Chimet and Engelhard. Analytical studies carried out according to conventional techniques have revealed the following structural differences between the two support materials. In particular, exemplary particles of the Chimet alumina had a BET surface area of 131 m²/g, whereas exemplary particles of the Engelhard HiQ (high quality) alumina had a BET surface area of 147 m²/g. Further, exemplary particles of the Chimet alumina included pores with an average diameter of 130 Angstrom, whereas exemplary particles of the Engelhard HiQ alumina included pores with an average diameter of 125 Angstrom. Thus, the surface area for the Engelhard catalyst is greater, and the pore size of the Chimet catalyst is greater. Either or both of these effects may influence the properties of the final catalyst.

Referring to FIGS. 5, 7, and 8, Al-1 and Al-2 represent Chimet and Engelhard alumina supports, respectively. The Engelhard support of FIG. 5 is 712-5-1566-1 alumina and the Engelhard support of FIG. 8 is Hi-Q 712A-5-1584-1 alumina. It can be seen from FIGS. 5, 7, and 8 that productivity varies in a more monotonic, or smooth, fashion with promoter content and with rhenium content for the catalysts including the Engelhard supports. Thus, it appears that activity, e.g. productivity, is more controllable, or predictable, for the catalysts including the Engelhard supports. In contrast, it can be seen from FIGS. 6 and 8 that, for lower rhenium content, e,g for 0.1, 0.3, and 0.5% rhenium, the productivity for the catalysts including the catalysts including the Chimet support is higher than the productivity for the catalysts of the same respective nominal compositions of cobalt, rhenium, and promoter and including the Hi-Q Engelhard support.

The catalysts of the preferred embodiments of the present invention may be prepared by any of the methods known to those skilled in the art. By way of illustration and not limitation, such methods include impregnating the catalytically active compounds or precursors onto a support, extruding one or more catalytically active compounds or precursors together with support material to prepare catalyst extrudates, and/or precipitating the catalytically active compounds or precursors onto a support.

The most preferred method of preparation may vary among those skilled in the art, depending for example on the desired catalyst particle size. Those skilled in the art are able to select the most suitable method for a given set of requirements.

One method of preparing a supported metal catalyst (e.g., a supported cobalt, cobalt/rhenium, or cobalt/rhenium/promoter catalyst) is by incipient wetness impregnation of the support with an aqueous solution of a soluble metal salt such as nitrate, acetate, acetylacetonate or the like. Another method of preparing a supported metal catalyst is by a melt impregnation technique, which involves preparing the supported metal catalyst from a molten metal salt. One preferred method is to impregnate the support with a molten metal nitrate (e.g., Co(NO₃)₂.6H₂O). Alternatively, the support can be impregnated with a solution of zero valent metal precursor. One preferred method is to impregnate the support with a solution of zero valent cobalt such as Co₂(CO)₈, Co₄(CO)₁₂ or the like in a suitable organic solvent (e.g., toluene). Suitable rhenium compounds are the common water soluble ones, e.g., rhenium heptoxide (Re₂O₇) and ammonium perrhenate (NH₄ReO₄). Likewise, suitable promoter compounds are the common water soluble ones, e.g. boria (B2O₃), manganese nitrate (Mn(NO₃)₂), phosphorous quintoxide P₂O₅), and ammonium vanadate (NH₄VO₃). Alternatively, a promoter compound may be acid soluble. Accordingly, an acid soluble promoter may be dissolved in acid and added to a water solution. In a preferred method, the impregnation of the support with a solution is accomplished by slurrying the support into the solution.

Thus, a preferred method of adding a promoter to a catalyst containing cobalt and rhenium includes slurrying a support into a solution containing cobalt, rhenium, and the promoter. In this method, the promoter is co-dispersed with cobalt on the support. An alternate method of adding a promoter to a catalyst containing cobalt and rhenium includes providing a supported cobalt catalyst followed by slurrying the supported cobalt catalyst into a solution containing rhenium and the promoter. In this method, the promoter is preferably layered over the cobalt.

The impregnated support is dried and reduced with hydrogen or a hydrogen containing gas. The hydrogen reduction step may not be necessary if the catalyst is prepared with zero valent cobalt. In another preferred method, the impregnated support is dried, oxidized with air or oxygen and reduced in the presence of hydrogen.

Typically, at least a portion of the metal(s) of the catalytic metal component (a) of the catalysts of the present invention is present in a reduced state (i.e., in the metallic state). Therefore, it is normally advantageous to activate the catalyst prior to use by a reduction treatment, in the presence of hydrogen at an elevated temperature. Typically, the catalyst is treated with hydrogen at a temperature in the range of from about 75° C. to about 500° C., for about 0.5 to about 24 hours at a pressure of about 1 to about 75 atm. Pure hydrogen may be used in the reduction treatment, as may a mixture of hydrogen and an inert gas such as nitrogen, or a mixture of hydrogen and other gases as are known in the art, such as carbon monoxide and carbon dioxide. Reduction with pure hydrogen and reduction with a mixture of hydrogen and carbon monoxide are preferred. The amount of hydrogen may range from about 1% to about 100% by volume.

The catalysts of the preferred embodiments of the present invention may be used in a Fischer-Tropsch process. It is preferred that the process yield hydrocarbons in a weight range suitable for processing to produce diesel fuel, such as C₁₁₊ hydrocarbons (11 or more carbon atoms per hydrocarbon chain.) Alternatively, the process may yield hydrocarbons in a weight range suitable for processing to produce gasoline, such as C₅₊ hydrocarbons (5 or more carbon atoms for hydrocarbon chain.)

The feed gases charged to the process of the invention comprise hydrogen, or a hydrogen source, and carbon monoxide. H₂/CO mixtures suitable as a feedstock for conversion to hydrocarbons according to the process of this invention can be obtained from light hydrocarbons such as methane by means of steam reforming, partial oxidation, or other processes known in the art. Preferably the hydrogen is provided by free hydrogen, although some Fischer-Tropsch catalysts have sufficient water gas shift activity to convert some water to hydrogen for use in the Fischer-Tropsch process. It is preferred that the molar ratio of hydrogen to carbon monoxide in the feed be greater than 0.5:1 (e.g., from about 0.67 to 2.5). Preferably, the feed gas stream contains hydrogen and carbon monoxide in a molar ratio of about 2:1. The feed gas may also contain carbon dioxide. The feed gas stream should contain a low concentration of compounds or elements that have a deleterious effect on the catalyst, such as poisons. For example, the feed gas may need to be pre-treated to ensure that it contains low concentrations of sulfur or nitrogen compounds such as hydrogen sulfide, ammonia and carbonyl sulfides.

The feed gas is contacted with the catalyst in a reaction zone. Mechanical arrangements of conventional design may be employed as the reaction zone including, for example, fixed bed, fluidized bed, slurry phase, slurry bubble column, reactive distillation column, or ebulliating bed reactors, among others, may be used. Accordingly, the size and physical form of the catalyst particles may vary depending on the reactor in which they are to be used.

The Fischer-Tropsch process is typically run in a continuous mode. In this mode, the gas hourly space velocity through the reaction zone typically may range from about 100 volumes/hour/volume catalyst (v/hr/v) to about 10,000 v/hr/v, preferably from about 300 v/hr/v to about 2,000 v/hr/v. The reaction zone temperature is typically in the range from about 160° C. to about 300° C. Preferably, the reaction zone is operated at conversion promoting conditions at temperatures from about 190° C. to about 260° C. The reaction zone pressure is typically in the range of about 80 psig (653 kPa) to about 1000 psig (6994 kPa), preferably, from 80 psig (653 kPa) to about 600 psig (4237 kPa), and still more preferably, from about 140 psig (1066 kPa) to about 400 psig (2858 kPa).

The products resulting from the process will have a great range of molecular weights. Typically, the carbon number range of the product hydrocarbons will start at methane and continue to the limits observable by modem analysis, about 50 to 100 carbons per molecule. The process is particularly useful for making hydrocarbons having five or more carbon atoms especially when the above-referenced preferred space velocity, temperature and pressure ranges are employed.

The wide range of hydrocarbons produced in the reaction zone will typically afford liquid phase products at the reaction zone operating conditions. Therefore, the effluent stream of the reaction zone will often be a mixed-phase stream including liquid and vapor phase products. The effluent stream may also include wax. The effluent stream of the reaction zone may be cooled to effect the condensation of additional amounts of hydrocarbons and passed into a vapor-liquid separation zone separating the liquid- and vapor-phase products. The vapor-phase material may be passed into a second stage of cooling for recovery of additional hydrocarbons. The liquid-phase material from the initial vapor-liquid separation zone together with any liquid from a subsequent separation zone may be fed into a fractionation column. Typically, a stripping column is employed first to remove light hydrocarbons such as propane and butane. The remaining hydrocarbons may be passed into a fractionation column, where they are separated by boiling point range into products such as naphtha, kerosene and fuel oils. Hydrocarbons recovered from the reaction zone and having a boiling point above that of the desired products may be passed into conventional processing equipment such as a hydrocracking zone in order to reduce their molecular weight. The gas phase recovered from the reactor zone effluent stream after hydrocarbon recovery may be partially recycled if it contains a sufficient quantity of hydrogen and/or carbon monoxide.

Without further elaboration, it is believed that one skilled in the art can, using the description herein, utilize the present invention to its fullest extent. The following embodiments are to be construed as illustrative, and not as constraining the scope of the present invention in any way whatsoever.

### EXAMPLES

Treatment of Catalyst Slurry

Each of the catalyst slurries described below was dried at 80° C. The solids were removed from the oven and exposed to air to absorb moisture. The solids were then dried again at 80° C. followed by heating the solids at 0.5° C. per minute to 350° C. and maintaining the solids at this temperature for 18 minutes. The solids were then heated at 0.5° C. per minute to 450° C., and reduced in hydrogen flow at 450° C. for 6 hours. The material was cooled and flushed with nitrogen overnight and then sealed for transport into an inert atmosphere glove box. The recovered catalyst was bottled and sealed for storage inside the glove box until Fischer-Tropsch testing could be completed.

Catalyst Reduction

Each of the catalyst samples used in continuous testing was treated with hydrogen insitu prior to use in the Fischer-Tropsch reaction in a fixed bed reactor. The catalyst sample was placed in the reactor the reactor and heated under 100 sccm (1.7×10⁻⁶ m³/s) hydrogen at 1° C./minute to 100° C. and held at 100° C. for one hour. The catalysts were then heated at 1° C./minute to 400° C. and held at 400° C. for four hours under 100 sccm (1.7×10⁻⁶ m³/s) hydrogen. The samples were cooled in hydrogen and purged with nitrogen before use.

General Procedure for Batch Testing

For the batch tests, a 2 mL pressure vessel was heated at 225° C. under 1000 psig (6994 kPa) of H₂:CO (2:1) and maintained at that temperature and pressure for 1 hour. In a typical run, roughly 20 mg of the reduced catalyst and 1 mL of n-octane was added to the vessel. After one hour, the reactor vessel was cooled in ice, vented, and an internal standard of di-n-butylether was added. The reaction product was analyzed on an HP6890 gas chromatograph. Hydrocarbons in the range of C₁₁-C₄₀ were analyzed relative to the internal standard. The lower hydrocarbons were not analyzed, since they are masked by the solvent and are also vented as the pressure is reduced. In the following examples, all catalyst having the same number designations but different letter designations were made on the same day and tested in the synthesis reaction under the same conditions.

A nominal composition was computed according to the amount by weight of alumina or fluorided alumina, the amount of elemental cobalt, the amount of any elemental rhenium, and the amount of any elemental promoter used to prepare the catalyst. Where a % is used in the nominal composition, it is a weight %.

A C₁₁₊ Productivity (g C₁₁₊/hour/kg catalyst) was calculated based on the integrated production of the C₁₁-C₄₀ hydrocarbons per kg of catalyst per hour. The logarithm of the weight fraction for each carbon number ln(Wₙ/n) was plotted as the ordinate vs. number of carbon atoms in (Wₙ/n) as the abscissa. From the slope, a value of alpha was obtained. The results of runs over a variety of catalysts at 225° C. are shown in Tables 1-8.

### EXAMPLES 1A-1C

### Boron Promoter

Re₂O₇ (0.0650 gm) and optionally B₂O₃ were dissolved in a small amount of water, added to molten Co(NO₃)₂.6H₂O (4.9383 gm) and mixed well to form a solution. B₂O₃ in the amount of θ gm, added, 0.0161 gm and 0.0805 gm was used for Examples 1A-1C, respectively. Alumina (Chimet) in the amount of 3.9500 gm, 3.9500 gm, and 3.9250 gm for Examples 1A-1C, respectively, was slurried into the solution. The slurry was treated according to the procedure described describe above.

The nominal composition for each catalyst is given in Table 1. Further, results for the C₁₁₊ productivity and for α, obtained by using the batch testing procedure described above for each catalyst is given in Table 1.

The results shown in Table 1 illustrate activity as a promoter for boron in the form of increased C₁₁₊ productivity as compared to a similar catalyst without added boron. The catalysts compared are similar in that they have a comparable wt. % of any other supported material, in particular cobalt and rhenium. Further, the results shown in Table 1 illustrate increased C₁₁₊ productivity for a catalyst including 0.5 wt % boron in the nominal composition, as compared to another similar catalyst including 0.1 wt % boron in the nominal composition.

TABLE 1

| Example | Nominal Composition | C₁₁₊ Productivity | Alpha |
|---|---|---|---|
| 1A | 20% Co/1% Re/Al2O3 | 690 | 0.89 |
| 1B | 20% Co/1% Re/0.1% B/Al2O3 | 820 | 0.89 |
| 1C | 20% Co/1% Re/0.5% B/Al2O3 | 960 | 0.89 |

### EXAMPLES 2A-3D

### Manganese Promoter

For Examples 2A-2D, NH₄ReO₄ (0.1440 gm) and optionally B₂O₃ were dissolved in a small amount of water, added to molten Co(NO₃)₂.6H₂O (4.9383 gm) and mixed well to form a solution. Mn(NO₃)₂ in the amount of 0 gm, 0.0160 gm, 0.0480 gm, and 0.161 gm was used for Examples 2A-2D, respectively. Alumina (Chimet) in the amount of 3.9000 gm, 3.9875 gm, 3.8925 gm, and 3.8750, for Examples 2A-2D, respectively, was slurried into the solution. The slurry was treated according to the procedure described describe above.

For Examples 3A-3D, the same procedure was followed, with the exception that Engelhard 712-5-1566-1 alumina was used in place of Chimet alumina.

The nominal composition for each catalyst is given in Table 2. Further, results for the C₁₁₊ productivity and for α, obtained by using the batch testing procedure described above for each catalyst is given in Table 2.

The results shown in Table 2 illustrate activity as a promoter for manganese in the form of increased C₁₁₊ productivity as compared to a similar catalyst without added manganese. The catalysts compared are similar in that they have a comparable wt. % of any other supported material, in particular cobalt and rhenium. Further, the results shown in Table 2 illustrate increased C₁₁₊ productivity for a catalyst including 0.3 wt % or 1% manganese in the nominal composition, as compared to another similar catalyst including 0.1 wt % manganese in the nominal composition. Thus, these results suggest that 0.3% wt. and 1 wt. % manganese are each more preferred to 0.1 wt. % manganese for these catalysts. Still further, these results suggest an optimal wt. % of manganese between 0.3 and 1.0 for these catalysts.

The results shown in Table 2 illustrate an increase in C₁₁₊ productivity for a manganese-promoted cobalt/rhenium catalyst of between about 3% and about 47%, depending on the composition.

TABLE 2

| Example | Nominal Composition | C₁₁₊ Productivity | Alpha |
|---|---|---|---|
| 2A | 20% Co/2% Re/Al2O3 | 800 | 0.9 |
| 2B | 20% Co/2% Re/0.1% Mn/Al2O3 | 830 | 0.89 |
| 2C | 20% Co/2% Re/0.3% Mn/Al2O3 | 1000 | 0.9 |
| 2D | 20% Co/2% Re/1% Mn/Al2O3 | 920 | 0.89 |
| 3A | 20% Co/2% Re/Al2O3 | 520 | 0.89 |
| 3B | 20% Co/2% Re/0.1% Mn/Al2O3 | 600 | 0.87 |
| 3C | 20% Co/2% Re/0.3% Mn/Al2O3 | 620 | 0.89 |
| 3D | 20% Co/2% Re/1% Mn/Al2O3 | 670 | 0.89 |

### EXAMPLES 4A-5C

### Phosporous Promoter

Re₂O₇ (0.0650 gm) and optionally P₂O₅ were dissolved in a small amount of water, added to molten Co(NO₃)₂.6H₂O (4.9384 gm) and mixed well to form a solution. P₂O₅ in the amount of θ gm, 0.0011 gm, 0.0055 gm, 0.0110 gm, 0.0550 gm, 0.1100 gm, 0.2200 gm. and 0.5500 gm was used for Examples 4A-4H, respectively. Alumina (Chimet) in the amount of 3.9490 gm, 3.9475, 3.9450, 3.9500, 3.9250, 3.9000, 3.8500, and 3.7000 gm for Examples 4A-4H, respectively, was slurried into the solution. The slurry was treated according to the procedure described describe above.

The nominal composition for each catalyst is given in Table 3. Further, results for the C₁₁₊ productivity and for α, obtained by using the batch testing procedure described above for each catalyst are given in Table 3.

For examples 5A, 5B, and 5C, the same catalysts as for Examples 4A, 4B, and 4D were testing using a modification of the batch testing procedure described above. In particular, 50 mg of catalyst was used and the catalyst was not added to a solvent, in particular n-octane was not used.

TABLE 3

| Example | Nominal Composition | C₁₁₊ Productivity | Alpha |
|---|---|---|---|
| 4A | 20% Co/1% Re/Al2O3 | 890 | 0.89 |
| 4B | 20% Co/1% Re/0.01% P/Al2O3 | 870 | 0.9 |
| 4C | 20% Co/1% Re/0.05% P/Al2O3 | 720 | 0.87 |
| 4D | 20% Co/1% Re/0.1% P/Al2O3 | 780 | 0.89 |
| 4E | 20% Co/1% Re/0.5% P/Al2O3 | 720 | 0.9 |
| 4F | 20% Co/1% Re/1% P/Al2O3 | 680 | 0.89 |
| 5A | 20% Co/1% Re/Al2O3 | 240 | 0.89 |
| 5B | 20% Co/1% Re/0.01% P/Al2O3 | 450 | 0.9 |
| 5C | 20% Co/1% Re/0.1% P/Al2O3 | 280 | 0.89 |

### EXAMPLES 6A-6D

### Phosporous and Manganese Promoters

Re₂O₇ (0.0650 gm), P₂O₅ (0.012115 gm), and optionally Mn(NO₃)₂ were dissolved in a small amount of water, added to molten Co(NO₃)₂.6H₂O (4.9384 gm) and mixed well to form a solution. Mn(NO₃)₂ in the amount of 0 gm, 0.0160 gm, 0.0480 gm, and 0.1610 gm was used for Examples 6A-6D, respectively. Alumina (Chimet) in the amount of 3.9450 gm, 3.9400 gm, 3.9300 gm, and 3.8950 gm for Examples 6A-6D, respectively, was slurried into the solution. The slurry was treated according to the procedure described describe above.

The nominal composition for each catalyst is given in Table 4. Further, results for the C₁₁₊ productivity and for α, obtained by using the batch testing procedure described above or each catalyst is given in Table 4.

The results shown in Table 4 illustrate that a favorable productivity may be achieved with a catalyst including a combination of promoters selecting according to the preferred embodiments of the present invention.

TABLE 4

| | | C₁₁₊ | |
|---|---|---|---|
| | | Product- | Al- |
| Example | Nominal Composition | ivity | pha |
| 6A | 20% Co/1% Re/0.1% P/Al2O3 | 790 | 0.88 |
| 6B | 20% Co/1% Re/0.1% P/0.1% Mn/Al2O3 | 640 | 0.88 |
| 6C | 20% Co/1% Re/0.1% P/0.3% Mn/Al2O3 | 730 | 0.89 |
| 6D | 20% Co/1% Re/0.1% P/1% Mn/Al2O3 | 710 | 0.89 |

### EXAMPLES 7A-7C

### Vanadium Promoter

Re₂O₇ (0.0650 gm) and optionally NH₄VO₃ were dissolved in a small amount of water, added to molten Co(NO₃)₂.6H₂O (4.9384 gm) and mixed well to form a solution. B₂O₃ in the amount of 0 gm, 0.0287 gm and 0.0574 gm was used for Examples 7A-7C, respectively. Alumina (Chimet) in the amount of 3.9500 gm, 3.9375 gm, and 3.9250 gm for Examples 7A-7C, respectively, was slurried into the solution. The slurry was treated according to the procedure described describe above.

The nominal composition for each catalyst is given in Table 5. Further, results for the C₁₁₊ productivity and for α, obtained by using the batch testing procedure described above for each catalyst is given in Table 5.

The results shown in Table 5 illustrate activity as a promoter for vanadium in the form of increased C₁₁₊ productivity as compared to a similar catalyst without added vanandium. The catalysts compared are similar in that they have a comparable wt. % of any other supported material, in particular cobalt and rhenium.

Further, the results shown in Table 5 illustrate an increase in C₁₁₊ productivity for a vanandium-promoted cobalt/rhenium catalyst of between about 6% and about 10%, depending on the composition.

TABLE 5

| Example | Nominal Composition | C₁₁₊ Productivity | Alpha |
|---|---|---|---|
| E93826- | 20% Co/1% Re/Al2O3 | 870 | 0.9 |
| 39A | | | |
| E93826- | 20% Co/1% Re/0.25% V/Al2O3 | 960 | 0.88 |
| 39B | | | |
| E93826- | 20% Co/1% Re/0.5% V/Al2O3 | 920 | 0.9 |
| 39C | | | |

### EXAMPLES 8A-12D

### Co-dispersed Promoter

### Chimet Alumina Support

For examples 6A-8D each promoter was co-mixed with cobalt and co-dispersed with cobalt on an alumina support. A series of catalyst were prepared, each catalyst in a single series having a nominal composition including a preselected weight percent of rhenium as a promoter and having an optional second promoter selected from boron, phosphorous, and manganese. Chimet alumina was used for the support.

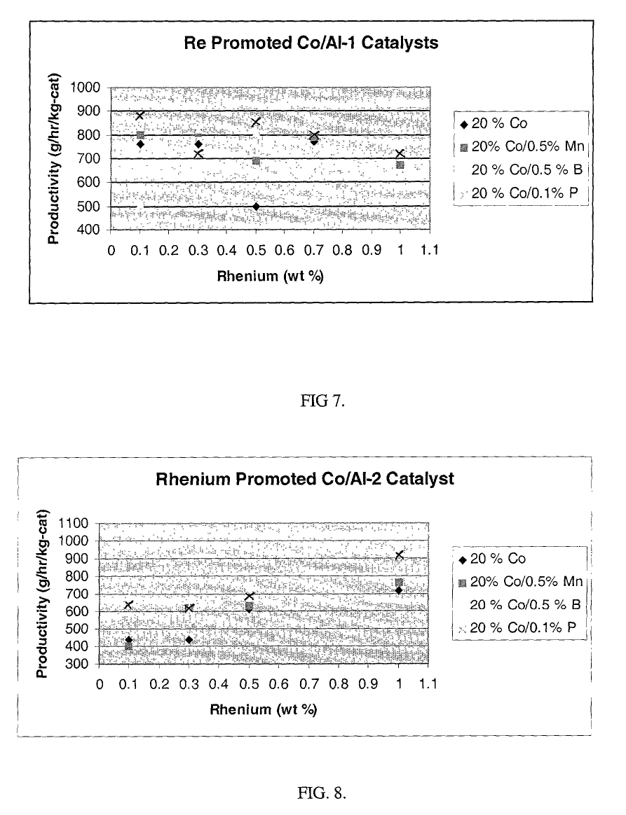

The nominal composition for each catalyst is given in Table 6. Further, results for the C₁₁₊ productivity and for α, obtained by using the batch testing procedure described above, for each catalyst is given in Table 6. The results for productivity are further illustrated in FIG. 7. Al-1 denotes the Chimet alumina support.

### EXAMPLES 8A-8C

### 1 Wt. % Rhenium

Re₂O₇ (0.0650 gm) and a second promoter precursor were dissolved in a small amount of water, added to molten Co(NO₃)₂.6H₂O (4.9383 gm) and mixed well to form a solution. The identities and amounts of the second promoter precursors were Mn(NO₃)₂ (0.1610 gm), B₂O₃ (0.0805 gm), and B₂O₃ (0.1610 gm) for Examples 8A-8C, respectively. Alumina in the amount of 3.9250 gm, 3.9250 gm, and 3.9000 gm for Examples 8A-8C, respectively, was slurried into the solution. The slurry was treated according to the procedure described describe above.

### EXAMPLES 9A-9D

### 0.7 Wt. % Rhenium

Re₂O₇ (0.0455 gm) and an optional second promoter precursor were dissolved in a small amount of water, added to molten Co(NO₃)₂.6H₂O (4.9383 gm) and mixed well to form a solution. The identities and amounts of the second promoter precursors were Mn(NO₃)₂ (0.1610 gm), and B₂O₃ (0.0805 gm), and P₂O₅ (0.0115 gm) for Examples 9B-9D, respectively. Alumina in the amount of 3.9650 gm, 3.9400 gm, 3.9400 gm, and 3.9600 gm for Examples 9A-9D, respectively, was slurried into the solution. The slurry was treated according to the procedure described describe above.

### EXAMPLES 10A-10D

### 0.5 Wt. % Rhenium

Re₂O₇ (0.0325 gm) and an optional second promoter precursor were dissolved in a small amount of water, added to molten Co(NO₃)₂.6H₂O (4.9383 gm) and mixed well to form a solution. The identities and amounts of the optional second promoter precursors were Mn(NO₃)₂ (0.1610 gm), and B₂O₃ (0.0805 gm), and P₂O₅ (0.0115 gm) for Examples 10BA-10D, respectively. Alumina in the amount of 3.975 gm, 3.9500 gm, 3.9500 gm, and 3.9700 gm for Examples 10A-10D, respectively, was slurried into the solution. The slurry was treated according to the procedure described describe above.

### EXAMPLES 11A-11D

### 0.3 Wt. % Rhenium

Re₂O₇ (0.0195 gm) and an optional second promoter precursor were dissolved in a small amount of water, added to molten Co(NO₃)₂.6H₂O (4.9383 gm) and mixed well to form a solution. The identities and amounts of the second promoter precursors were Mn(NO₃)₂ (0.1610 gm), and B₂O₃ (0.0805 gm), and P₂O₅ (0.0115 gm) for Examples 11B-11D, respectively. Alumina in the amount of 3.9850 gm, 3.9600 gm, 3.9600 gm, and 3.9800 gm for Examples 11A-11D, respectively, was slurried into the solution. The slurry was treated according to the procedure described describe above.

### EXAMPLES 12A-12D

### 0.1 Wt. Rhenium

Re₂O₇ (0.0065 gm) and an optional second promoter precursor were dissolved in a small amount of water, added to molten Co(NO₃)₂.6H₂O (4.9383 gm) and mixed well to form a solution. The identities and amounts of the second promoter precursors were Mn(NO₃)₂ (0.1610 gm), and B₂O₃ (0.0805 gm), and P₂O₅ (0.0115 gm) for Examples 12B-12D, respectively. Alumina in the amount of 3.9850 gm, 3.9600 gm, 3.9600 gm, and 3.9800 gm for Examples 12A-12D, respectively, was slurried into the solution. The slurry was treated according to the procedure described describe above.

TABLE 6

| | | C₁₁₊ Product- | |
|---|---|---|---|
| Example | Nominal Composition | ivity | Alpha |
| 8A | 20% Co/1% Re/0.5% Mn/Al2O3 | 690 | 0.9 |
| 8B | 20% Co/1% Re/0.5% B/Al2O3 | 740 | 0.9 |
| 8C | 20% Co/1% Re/1% B/Al2O3 | 720 | 0.9 |
| 9A | 20% Co/0.7% Re/Al2O3 | 770 | 0.9 |
| 9B | 20% Co/0.7% Re/0.5% Mn/Al2O3 | 790 | 0.9 |
| 9C | 20% Co/0.7% Re/0.5% B/Al2O3 | 530 | 0.89 |
| 9D | 20% Co/0.7% Re/0.1% P/Al2O3 | 800 | 0.88 |
| 10A | 20% Co/0.5% Re/Al2O3 | 500 | 0.9 |
| 10B | 20% Co/0.5% Re/0.5% Mn/Al2O3 | 690 | 0.9 |
| 10C | 20% Co/0.5% Re/0.5% B/Al2O3 | 810 | 0.91 |
| 10D | 20% Co/0.5% Re/0.1% P/Al2O3 | 850 | 0.9 |
| 11A | 20% Co/0.3% Re/Al2O3 | 760 | 0.9 |
| 11B | 20% Co/0.3% Re/0.5% Mn/Al2O3 | 810 | 0.9 |
| 11C | 20% Co/0.3% Re/0.5% B/Al2O3 | 820 | 0.9 |
| 11D | 20% Co/0.3% Re/0.1% P/Al2O3 | 720 | 0.9 |
| 12A | 20% Co/0.1% Re/Al2O3 | 760 | 0.89 |
| 12B | 20% Co/0.1% Re/0.5% Mn/Al2O3 | 800 | 0.9 |
| 12C | 20% Co/0.1% Re/0.5% B/Al2O3 | 510 | 0.9 |
| 12D | 20% Co/0.1% Re/0.1% P/Al2O3 | 880 | 0.9 |

### EXAMPLES 13A-16D

### Co-dispersed Promoter

### Engelhard Alumina Support

For examples 13A-16D each promoter was co-mixed with cobalt and co-dispersed with cobalt on an alumina support. A series of catalysts were prepared, each catalyst in a single series having a nominal composition including a preselected weight percent of rhenium as a promoter and having an optional second promoter selected from boron, phosphorous, and manganese. Engelhard Hi Q: ENG-712A-5-1584-1 alumina was used for the support.

The nominal composition for each catalyst is given in Table 7. Further, results for the C₁₁₊ productivity and for α, obtained by using the batch testing procedure described above, for each catalyst is given in Table 7.

The results shown in Table 7 illustrate improved C₁₁⁺ productivity for a promoted cobalt-containing catalyst that contains rhenium as another promoter as compared to the same promoted cobalt-containing catalyst to which rhenium is not added.

Further, the results shown in Table 7 illustrate promoter activity in the form of improved C₁₁⁺ productivity for each of boron, phosphorous, and manganese.

Still further, the results shown in Table 7 illustrate a trend of decreasing C₁₁⁺ productivity as the second promoter is varied from boron to phosphorous to manganese.

The results for productivity are further illustrated in FIG. 8. Al-2 denotes the Engelhard alumina support.

### EXAMPLES 13A-13E

### 1 Wt. % Rhenium

For Example 13A, alumina (4.0000 gm) was slurried into molten Co(NO₃)₂.6H₂O (4.9383 gm). The slurry was treated according to the procedure described above.

For Examples, 13B-13E, Re₂O₇ (0.0650 gm) and a second promoter precursor were dissolved in a small amount of water, added to molten Co(NO₃)₂.6H₂O (4.9383 gm) and mixed well to form a solution. The identities and amounts of the second promoter precursors were Mn(NO₃)₂ (0.1610 gm), and B₂O₃ (0.0805 gm), and P₂O₅ (0.0115 gm) for Examples 13C-13E, respectively. Alumina in the amount of 3.9500 gm, 3.9250 gm, 3.9250 gm, and 3.9450 gm for Examples 13B-13E, respectively, was slurried into the solution. The slurry was treated according to the procedure described describe above.

### EXAMPLES 14A-14D

### 0.5 Wt. % Rhenium

Re₂O₇ (0.0325 gm) and a optional second promoter precursor were dissolved in a small amount of water, added to molten Co(NO₃)₂.6H₂O (4.9383 gm) and mixed well to form a solution. The identities and amounts of the second promoter precursors were Mn(NO₃)₂ (0.1610 gm), and B₂O₃ (0.0805 gm), and P₂O₅ (0.0115 gm) for Examples 14B-14D, respectively. Alumina in the amount of 3.975 gm, 3.9500 gm, 3.9500 gm, and 3.9700 gm for Examples 14A-14D, respectively, was slurried into the solution. The slurry was treated according to the procedure described describe above.

### EXAMPLES 15A-15D

### 0.3 Wt. % Rhenium

Re₂O₇ (0.0195 gm) and a optional second promoter precursor were dissolved in a small amount of water, added to molten Co(NO₃)₂.6H₂O (4.9383 gm) and mixed well to form a solution. The identities and amounts of the second promoter precursors were Mn(NO₃)₂ (0.1610 gm), and B₂O₃ (0.0805 gm), and P₂O₅ (0.0115 gm) for Examples 15B-1 5D, respectively. Alumina in the amount of 3.9850 gm, 3.9600 gm, 3.9600 gm, and 3.9800 gm for Examples 15A-15D, respectively, was slurried into the solution. The slurry was treated according to the procedure described describe above.

### EXAMPLES 16A-16D

### 0.1 Wt. % Rhenium

Re₂O₇ (0.0065 gm) and a optional second promoter precursor were dissolved in a small amount of water, added to molten Co(NO₃)₂.6H₂O (4.9383 gm) and mixed well to form a solution. The identities and amounts of the second promoter precursors were Mn(NO₃)₂ (0.1610 gm), and B₂O₃ (0.0805 gm), and P₂O₅ (0.0115 gm) for Examples 16B-16D, respectively. Alumina in the amount of 3.9850 gm, 3.9600 gm, 3.9600 gm, and 3.9800 gm for Examples 16A-16D, respectively, was slurried into the solution. The slurry was treated according to the procedure described describe above.

TABLE 7

| | | C₁₁₊ Product- | |
|---|---|---|---|
| Example | Nominal Composition | ivity | Alpha |
| 13A | 20% Co/Al2O3 | 670 | 0.9 |
| 13B | 20% Co/1% Re/Al2O3 | 720 | 0.9 |
| 13C | 20% Co/1% Re/0.5% Mn/Al2O3 | 760 | 0.9 |
| 13D | 20% Co/1% Re/0.5% B/Al2O3 | 970 | 0.9 |
| 13E | 20% Co/1% Re/0.1% P/Al2O3 | 910 | 0.91 |
| 14A | 20% Co/0.5% Re/Al2O3 | 610 | 0.9 |
| 14B | 20% Co/0.5% Re/0.5% Mn/Al2O3 | 630 | 0.89 |
| 14C | 20% Co/0.5% Re/0.5% B/Al2O3 | 740 | 0.89 |
| 14D | 20% Co/0.5% Re/0.1% P/Al2O3 | 680 | 0.9 |
| 15A | 20% Co/0.3% Re/Al2O3 | 540 | 0.9 |
| 15B | 20% Co/0.3% Re/0.5% Mn/Al2O3 | 620 | 0.9 |
| 15C | 20% Co/0.3% Re/0.5% B/Al2O3 | 630 | 0.89 |
| 15D | 20% Co/0.3% Re/0.1% P/Al2O3 | 610 | 0.89 |
| 16A | 20% Co/0.1% Re/Al2O3 | 440 | 0.9 |
| 16B | 20% Co/0.1% Re/0.5% Mn/Al2O3 | 400 | 0.9 |
| 16C | 20% Co/0.1% Re/0.5% B/Al2O3 | 680 | 0.9 |
| 16D | 20% Co/0.1% Re/0.1% P/Al2O3 | 640 | 0.9 |

### EXAMPLES 19A-G

### Layered Promoter

For Examples 19A-G, at least one promoter was added to a cobalt-containing catalyst supported on alumina, producing a promoter layer over a layer of cobalt. Pairs of catalyst compositions were prepared. Each pair included corresponding first and second catalysts, each corresponding catalyst having a nominal composition including the same preselected weight percent of rhenium. Each first catalyst had a promoter layer made of rhenium. In contrast, each corresponding second catalyst had a promoter layer made of rhenium co-mixed with a second promoter select from boron, phosphorous, and manganese.

For catalysts 19A-19G, precursors were added to a small amount of water. The identities and amounts of the precursors were Re₂O₃ (0.0130 gm) or Examples 19B, 19C, 19E, and 19G; B₂O₃ (0.0161 gm) for Examples 19A and 19C; P₂O₅ (0.0110 gm) for Examples 19D and 19E, and Mn(NO₃)₂ (0.0096 gm) for Examples 19F and 19G.

20%Co/Alumina (Engelhard 6568-9-11) was slurried into the solution in the amount of 0.9900 gm for Examples 19A-B, 0.9850 gm for Examples 19C, 19E, and 19F, 0.9950 gm for Example 19D, and 0.9780 gm for Example 19G. The slurry was treated according to the procedure described above.

The nominal composition for each catalyst is given in Table 8. Further, results for the C₁₁₊ productivity and for α, obtained by using the batch testing procedure described above for each catalyst is given in Table 8.

A comparison of Example 19C with 19A, Example 19E with 19D, and Example 19G with 19F, in Table 8, illustrates improved C₁₁⁺ productivity for a promoted cobalt-containing catalyst that contains rhenium as another promoter as compared to the same promoted cobalt-containing catalyst to which rhenium is not added.

TABLE 8

| Example | Nominal Composition | C₁₁₊ Productivity | Alpha |
|---|---|---|---|
| 19A | 20% Co/0.5% B/Al2O3 | 600 | 0.89 |
| 19B | 20% Co/1% Re/Al2O3 | 880 | 0.88 |
| 19C | 20% Co/1% Re/0.5% B/Al2O3 | 770 | 0.89 |
| 19D | 20% Co/0.5% P/Al2O3 | 590 | 0.89 |
| 19E | 20% Co/1% Re/0.5% P/Al2O3 | 760 | 0.89 |
| 19F | 20% Co/0.3% Mn/Al2O3 | 760 | 0.88 |
| 19G | 20% Co/1% Re/0.3% Mn/Al2O3 | 860 | 0.9 |

### EXAMPLES 20A-23C

### Fluorided Alumina Support

For Examples 20A-23C, Engelhard 4352 (6541-18-1) was used for the fluorided alumina. The nominal composition for each catalyst is given in Table 10. Further, results for the C₁₁₊ productivity and for α, obtained by using the batch testing procedure described above, for each catalyst is given in Table 9.

### EXAMPLES 20A-20D

### Boron Promoter

Re₂O₇ (0.0650 gm) and a variable amount of B₂O₃ were dissolved in a small amount of water, added to molten Co(NO₃)₂.6H₂O (4.9384 gm) and mixed well to form a solution. The variable amount of B₂O₃ as 0 gm, 0.0016 gm, 0.0080 gm, and 0.0161 gm for Examples 20A-20D, respectively. Fluorided alumina was slurried into the solution in the amount of 3.9500 gm, 3.9495 gm, 3.9475 gm, and 3.9450 gm. The slurry was treated as describe above.

### EXAMPLES 21A-21C

### Boron Promoter

Re₂O₇ (0.0650 gm) and a variable amount of B₂O₃ were dissolved in a small amount of water, added to molten Co(NO₃)₂.6H₂O (4.9383 gm) and mixed well to form a solution. The variable amount of B₂O₃ was none, 0.0161 gm, and 0.0805 gm for Examples 21A-21C, respectively. Fluorided alumina was slurried into the solution, in the amount of 3.9500 gm, 3.9450 gm, and 3.9250 gm for Examples 21A-21C, respectively. The slurry was treated as described above.

### EXAMPLES 22A-22C

### Manganese Promoter

NH₄ReO₄ (0.0720 gm) and a variable amount of Mn(NO₃)₂ were dissolved in a small amount of water, added to molten Co(NO₃)₂.6H₂O (4.9383 gm) and mixed well to form a solution. The variable amount of Mn(NO₃)₂ was 0.0323 gm, 0.0968 gm, and 0.3226 gm for Examples 22A-22C, respectively. Fluorided alumina was slurried into the solution, in the amount of 3.9450 gm, 3.9350 gm, and 3.9000 gm for Examples 22A-22C, respectively. The slurry was treated as described above.

### EXAMPLES 23A-23C

### Phosphorous Promoter

Re₂O₇ (0.0650 gm) and a variable amount of P₂O₅ were dissolved in a small amount of water, added to molten Co(NO₃)₂.6H₂O (4.9383 gm) and mixed well to form a solution. The variable amount of P₂O₅ was 0.0573, 0.1146, and 0.2291 for Examples 23A-23C, respectively. Fluorided alumina was slurried into the solution, in the amount of 3.9250 gm, 3.9000 gm, and 3.8500 gm for Examples 23A-23C, respectively.

TABLE 9

| | | C₁₁₊ Pro- | |
|---|---|---|---|
| Example | Nominal Composition | ductivity | Alpha |
| 20A | 20% Co/1% Re/Al2O3(F) | 790 | 0.88 |
| 20B | 20% Co/1% Re/0.01% B/Al2O3(F) | 750 | 0.89 |
| 20C | 20% Co/1% Re/0.05% B/Al2O3(F) | 750 | 0.88 |
| 20D | 20% Co/1% Re/0.1% B/Al2O3(F) | 710 | 0.88 |
| 21A | 20% Co/1% Re/Al2O3(F) | 580 | 0.89 |
| 21B | 20% Co/1% Re/0.1% B/Al2O3(F) | 610 | 0.89 |
| 21C | 20% Co/1% Re/0.5% B/Al2O3(F) | 720 | 0.89 |
| 22A | 20% Co/1% Re/0.1% Mn/Al2O3(F) | 650 | 0.9 |
| 22B | 20% Co/1% Re/0.3% Mn/Al2O3(F) | 590 | 0.89 |
| 22C | 20% Co/1% Re/1% Mn/Al2O3(F) | 560 | 0.88 |
| 23A | 20% Co/1% Re/0.5% P/Al2O3(F) | 560 | 0.89 |
| 23B | 20% Co/1% Re/1% P/Al2O3(F) | 540 | 0.89 |
| 23C | 20% Co/1% Re/2% P/Al2O3(F) | 510 | 0.89 |

### EXAMPLES 25A-27C

### Alkali Promoter

Lithium and potassium promoted catalysts were tested as exemplary of alkali promoters, including alkali metals from Group 1 of the periodic table. Group 1 alkali metals include lithium, sodium, potassium, rubidium, cesium, and francium.

### EXAMPLES 24A-24D

### Lithium Promoter

Re₂O₇ (0.0650 gm) and optionally Li₂O were dissolved in a small amount of water, added to molten Co(NO₃)₂.6H₂O (4.9384 gm) and mixed well to form a solution. Li₂O in the amount of 0 gm, 0.0054 gm, 0.0108 gm, and 0.0161 gm was used for Examples 24A-24D, respectively. Alumina (Chimet) in the amount of 3.9500 gm, 3.9475 gm, 3.9450 gm, and 3.9425 gm for Examples 24A-24D, respectively, was slurried into the solution. The slurry was treated according to the procedure described describe above.

The nominal composition for each catalyst is given in Table 10. Further, results for the C₁₁₊ productivity and for α, obtained by using the batch testing procedure described above for each catalyst is given in Table 10.

TABLE 10

| Example | Nominal Composition | C₁₁₊ Productivity | Alpha |
|---|---|---|---|
| 24A | 20% Co/1% Re/Al2O3 | 970 | 0.89 |
| 24B | 20% Co/1% Re/0.05% Li/Al2O3 | 830 | 0.9 |
| 24C | 20% Co/1% Re/0.1% Li/Al2O3 | 810 | 0.89 |
| 24D | 20% Co/1% Re/0.15% Li/Al2O3 | 800 | 0.89 |

### EXAMPLES 25A-27C

### Potassium Promoter

For examples 25A-25D, NH₄ReO₄ (0.1080) and optionally KNO₃ were dissolved in a small amount of water, added to molten Co(NO₃)₂.6H₂O (4.9384 gm) and mixed well to form a solution. KNO₃ in the amount of 0 gm, 0.0065 gm, 0.0195 gm, and 0.0645 gm was used for Examples 25A-25D, respectively. Alumina (Chimet) in the amount of 3.9250 gm, 3.9250 gm, 3.8875 gm, and 3.8500 gm for Examples 25A-25D, respectively, was slurried into the solution. The slurry was treated according to the procedure described describe above. Examples 26A and 26B are a retest of examples 25B and 25C.

For examples 27A-27C, NH₄ReO₄ (0.1440) and optionally KNO₃ were dissolved in a small amount of water, added to molten Co(NO₃)₂.6H₂O (4.9384 gm) and mixed well to form a solution. KNO₃ in the amount of none added, 0.0065 gm, and 0.0195 gm was used for Examples 26A-26C, respectively. Alumina (Engelhard 712-5-1566-1) in the amount of 3.9250, 3.9225, 3.8875, and 3.8500 gm for Examples 26A-26C, respectively, was slurried into the solution. The slurry was treated according to the procedure described describe above.

The nominal composition for each catalyst is given in Table 11. Further, results for the C₁₁₊ productivity and for α, obtained by using the batch testing procedure described above for each catalyst are given in Table 11.

TABLE 11

| | | C₁₁₊ | |
|---|---|---|---|
| Example | Nominal Composition | Productivity | Alpha |
| 25A | 20% Co/1.5% Re/Al2O3 | 740 | 0.9 |
| 25B | 20% Co/1.5% Re/0.05% K/Al2O3 | 530 | 0.9 |
| 25C | 20% Co/1.5% Re/0.15% K/Al2O3 | 620 | 0.89 |
| 25D | 20% Co/1.5% Re/0.5% K/Al2O3 | 350 | 0.88 |
| 26A | 20% Co/1.5% Re/0.05% K/Al2O3 | 740 | 0.9 |
| 26B | 20% Co/1.5% Re/0.15% K/Al2O3 | 480 | 0.89 |
| 27A | 20% Co/2% Re/Al2O3 | 770 | 0.88 |
| 27B | 20% Co/2% Re/0.05% K/Al2O3 | 470 | 0.87 |
| 27C | 20% Co/2% Re/0.15% K/Al2O3 | 440 | 0.88 |

General Procedure for Continuous Tests

The catalyst testing unit was composed of a syngas feed system, a tubular reactor, which had a set of wax and cold traps, back pressure regulators, and three gas chromatographs (one on-line and two off-line).

The carbon monoxide was purified before being fed to the reactor over a 22% lead oxide on alumina catalyst placed in a trap to remove any iron carbonyls present. The individual gases or mixtures of the gases were mixed in a 300 mL vessel filled with glass beads before entering the supply manifold feeding the reactor.

The reactor was made of ⅜ in. (0.95 cm) O.D. by ¼ in. (0.63 cm) I.D. stainless steel tubing. The length of the reactor tubing was 14 in. (35.6 cm). The actual length of the catalyst bed was 10 in. (25.4 cm) with 2 in. (5.1 cm) of 25/30 mesh (0.71/0.59 mm) glass beads and glass wool at the inlet and outlet of the reactor.

The wax and cold traps were made of 75 mL pressure cylinders. The wax traps were set at 140° C. while the cold traps were set at 0° C. The reactor had two wax traps in parallel followed by two cold traps in parallel. At any given time products from the reactor flowed through one wax and one cold trap in series. Following a material balance period, the hot and cold traps used were switched to the other set in parallel, if needed. The wax traps collected a heavy hydrocarbon product distribution (usually between C₆ and above) while the cold traps collected a lighter hydrocarbon product distribution (usually between C₃ and C₂₀). Water, a major product of the Fischer-Tropsch process was collected in both the traps.

General Analytical Procedure

The uncondensed gaseous products from the reactors were analyzed using a common on-line HP Refinery Gas Analyzer. The Refinery Gas Analyzer was equipped with two thermal conductivity detectors and measured the concentrations of CO, H₂, N₂, CO₂, CH₄, C₂ to C₅ alkenes/alkanes/isomers and water in the uncondensed reactor products. The products from each of the hot and cold traps were separated into an aqueous and an organic phase. The organic phase from the hot trap was usually solid at room temperature. A portion of this solid product was dissolved in carbon disulfide before analysis. The organic phase from the cold trap was usually liquid at room temperature and was analyzed as obtained. The aqueous phase from the two traps was combined and analyzed for alcohols and other oxygenates. Two off-line gas chromatographs equipped with flame ionization detectors were used for the analysis of the organic and aqueous phases collected from the wax and cold traps.

Catalyst Testing Procedure

Catalyst (3 g) to be tested was mixed with 4 grams of 25/30 mesh (0.71/0.59 mm) and 4 grams of 2 mm glass beads. The 14 in. (35.6 cm) tubular reactor was first loaded with 25/30 mesh (0.71/0.59 mm) glass beads so as to occupy 2 in. (5.1 cm) length of the reactor. The catalyst/glass bead mixture was then loaded and occupied 10 in. (25.4 cm) of the reactor length. The remaining 2 in. (5.1 cm) of reactor length was once again filled with 25/30 mesh (0.71/0.59 mm) glass beads. Both ends of the reactor were plugged with glass wool.

Catalyst activation was subsequently carried out using the following procedure. The reactor was heated to 120° C. under nitrogen flow (100 cc/min and 40 psig (377 kPa)) at a rate of 1.5° C./min. The reactor was maintained at 120° C. under these conditions for two hours for drying of the catalyst. At the end of the drying period, the flow was switched from nitrogen to hydrogen. The reactor was heated under hydrogen flow (100 cc/min and 40 psig (377 kPa)) at a rate of 1.4° C./min. to 350° C. The reactor was maintained at 350° C. under these conditions for sixteen hours for catalyst reduction. At the end of the reduction period, the flow was switched back to nitrogen and the reactor cooled to reaction temperature (usually 220° C.).

The reactor was pressurized to the desired reaction pressure and cooled to the desired reaction temperature. Syngas, with a 2:1 H₂/CO ratio was then fed to the reactor when reaction conditions were reached.

The first material balance period started at about four hours after the start of the reaction. A material balance period lasted for between 16 to 24 hours. During the material balance period, data was collected for feed syngas and exit uncondensed gas flow rates and compositions, weights and compositions of aqueous and organic phases collected in the wax and cold traps, and reaction conditions such as temperature and pressure. The information collected was then analyzed to get a total as well as individual carbon, hydrogen and oxygen material balances. From this information, CO Conversion (%), Selectivity/Alpha plot for all (C₁ to C₄₀) of the hydrocarbon products, C₅+ Productivity (g/hr/kg cat), weight percent CH₄ in hydrocarbon products (%) and other desired reactor outputs were calculated.

Catalyst Preparation

Catalyst samples used for continuous testing were, except for Examples 28-31 and 55-57, prepared from the catalysts of which samples were taken for batch testing, as described above. The correspondence is given in Table 12.

TABLE 12

Continuous
Testing Ex-

| Batch Testing Example No. | ample No.'s | |
|---|---|---|
| 13A | 27 | 54 |
| 13B | 31 | 58 |
| 11C | 32 | 59 |
| 10C | 33 | 60 |
| 1B | 34 | 61 |
| 1C | 35 | 62 |
| 2B | 36 | 63 |
| 2C | 37 | 64 |
| 2D | 38 | 65 |
| 3B | 39 | 66 |
| 13C | 40 | 68 |
| 4B | 41 | 69 |
| 4C | 44 | 70 |
| 4D | 43 | 71 |
| 4E | 44 | 72 |
| 4F | 45 | 73 |
| 7B | 46 | 74 |
| 20A | 47 | 75 |
| 20B | 48 | 76 |
| 20C | 49 | 77 |
| 20D | 50 | 78 |
| 24B | 51 | 79 |
| 24C | 52 | 80 |
| 25B | 53 | 81 |

The catalyst for Examples 28 and 55, the catalyst Examples 29 and 56, and the catalyst for Examples 30 and 57 were prepared by the same procedure as for Example 13B, except that Chimet alumina was used for the first two of these catalysts (Examples 28, 55, 29, 56) and Engelhard alumina (712A-5-1566-1) was used for the third of these catalysts (Examples 30, 55).

The results obtained from the continuous-flow Fischer-Tropsch catalyst testing unit is shown in Tables 13 and 14.

Table 13 lists the catalyst composition, CO Conversion (%), Alpha value from the Anderson-Shultz-Flory plot of the hydrocarbon product distribution, C₅+ Productivity (g C₅+/hour/catalyst) and weight percent methane in the total hydrocarbon product (%). The data were obtained during the first material balance period.

The temperature was 220° C., the pressure was between 340 psig (2445 kPa) to 362 (2597 kPa) and the space velocity was 2 NL/hour/g. cat. for all the examples in Table 13.

TABLE 13

| | | CO | | C5+ | |
|---|---|---|---|---|---|
| | | Conv. | | Productivity | Cl |
| Example | Catalyst Nominal Composition | (%) | Alpha | (g/h/kgcat) | (wt. %) |
| 27 | 20% Co/Al2O3 | 65 | 0.88 | 170 | 18 |
| 28 | 20% Co/1% Re/Al2O3 | 100 | 0.89 | 270 | 13 |
| 20 | 20% Co/1.0% ReAl2O3 | 99 | 0.90 | 260 | 9 |
| 30 | 20% Co/1.0% Re/Al2O3 | 100 | 0.89 | 240 | 15 |
| 31 | 20% Co/1% Re/Al2O3 | 77 | 0.89 | 280 | 15 |
| 32 | 20% Co/0.3% Re/0.5% B/Al2O3 | 100 | 0.89 | 270 | 2 |
| 33 | 20% Co/0.5% Re/0.5% B/Al2O3 | 100 | 0.89 | 280 | 2 |
| 34 | 20% Co/1.0% Re/0.1% B/Al2O3 | 100 | 0.90 | 250 | 15 |
| 35 | 20% Co/1.0% Re/0.5% B/Al2O3 | 100 | 0.91 | 250 | 18 |
| 36 | 20% Co,2% Re,0.1% Mn/Al2O3 | 100 | 1.04 | 280 | 13 |
| 37 | 20% Co/2.0% Re/0.3% Mn/Al2O3 | 100 | 0.90 | 220 | 17 |
| 38 | 20% Co/2.0% Re/1.0% Mn/Al2O3 | 100 | 0.90 | 200 | 23 |
| 39 | 20% Co/2% Re/0.1% Mn/Al2O3 | 89 | 0.90 | 280 | 3 |
| 40 | 20% Co/1.0% Re/0.5% Mn/Al2O3 | 82 | 0.89 | 240 | 13 |
| 41 | 20% Co/1.0% Re/0.01% P/Al2O3 | 100 | 0.89 | 240 | 22 |
| 42 | 20% Co/1.0% Re/0.05% P/Al2O3 | 100 | 0.90 | 270 | 19 |
| 43 | 20% Co/1.0% Re/0.1% P/Al2O3 | 98 | 0.91 | 280 | 17 |
| 44 | 20% Co/1.0% Re/0.5% P/Al2O3 | 95 | 0.88 | 230 | 14 |
| 45 | 20% Co/1% Re/1% P/Al2O3 | 81 | 0.90 | 230 | 13 |
| 46 | 20% Co/0.25% V/Al2O3 | 81 | 0.90 | 230 | 13 |
| 47 | 20% Co/1.0% Re/Al2O3(F) | 90 | 0.86 | 220 | 14 |
| 48 | 20% Co/1.0% Re/0.01% B/Al2O3(F) | 89 | 0.85 | 150 | 27 |
| 49 | 20% Co/1.0% Re/0.05% B/Al2O3(F) | 70 | 0.87 | 150 | 21 |
| 50 | 20% Co/1.0% Re/0.1% B/Al2O3(F) | 88 | 0.87 | 200 | 18 |
| 51 | 20% Co/1.0% Re/0.05% Li/Al2O3 | 100 | 0.87 | 140 | 32 |
| 52 | 20% Co/1.0% Re/0.05% Li/Al2O3 | 87 | 0.87 | 100 | 26 |
| 53 | 20% Co/1.5% Re/0.05% K/Al2O3 | 98 | 0.72 | 280 | 9 |

Table 14 lists the catalyst composition, CO Conversion (%), Alpha value from the Anderson-Shultz-Flory plot of the hydrocarbon product distribution, C₅+ Productivity (g C₅+/hour/catalyst) and weight percent methane in the total hydrocarbon product (%) for examples illustrating the extended lifetimes of the catalysts of the present invention. The data for each catalyst listed in Table 14 was obtained in the same run as for the same catalyst as listed in Table 14.

The data in Table 14 illustrates the superior results obtained using the catalysts of the present invention after approximately 160 hours of continuous operation. The temperature was 220° C., the pressure was between 340 psig (2445 kPa) to 362 (2597 kPa) and the space velocity was 2 NL/hour/g. cat. for all the examples in Table 13, i.e., the same conditions as for examples 27-53, for the length of operation.

Further, the data in Table 14 illustrates increased C₅₊ productivity and CO conversion for promoted catalysts as compared to similar unpromoted catalysts.

TABLE 14

| | | CO | | C5+ | |
|---|---|---|---|---|---|
| | | Conv. | | Productivity | Cl |
| Example | Catalyst | (%) | Alpha | (g/h/kgcat) | (wt. %) |
| 54 | 20% Co/Al2O3 | 56 | 0.87 | 150 | 17 |
| 55 | 20% Co/1.0% Re/Al2O3 | 81 | 0.89 | 210 | 11 |
| 56 | 20% Co/1% Re/Al2O3 | 77 | 0.88 | 220 | 12 |
| 57 | 20% Co/1.0% Re/Al2O3 | 94 | 0.90 | 180 | 12 |
| 58 | 20% Co/1.0% Re/Al2O3 | 75 | 0.89 | 230 | 15 |
| 59 | 20% Co/0.3% Re/0.5% B/Al2O3 | 100 | 0.88 | 120 | 2 |
| 60 | 20% Co/0.5% Re/0.5% B/Al2O3 | 99 | 0.89 | 300 | 2 |
| 61 | 20% Co/1.0% Re/0.1% B/Al2O3 | 87 | 0.90 | 270 | 11 |
| 62 | 20% Co/1.0% Re/0.5% B/Al2O3 | 98 | 0.90 | 280 | 14 |
| 63 | 20% Co/2% Re/0.1% Mn/Al2O3 | 97 | 0.92 | 260 | 10 |
| 64 | 20% Co/2.0% Re/0.3% Mn/Al2O3 | 92 | 0.91 | 360 | 11 |
| 65 | 20% Co/2.0% Re/1.0% Mn/Al2O3 | 100 | 0.90 | 270 | 17 |
| 66 | 20% Co/2% Re/0.1% Mn/Al2O3 | 94 | 0.89 | 280 | 15 |
| 67 | 20% Co/1.0% Re/0.5% Mn/Al2O3 | 82 | 0.90 | 230 | 17 |
| 68 | 20% Co/1.0% Re/0.01% P/Al2O3 | 89 | 0.88 | 210 | 17 |
| 69 | 20% Co/1.0% Re/0.05% P/Al2O3 | 90 | 0.89 | 270 | 15 |
| 70 | 20% Co/1.0% Re/0.1% P/Al2O3 | 84 | 0.90 | 270 | 12 |
| 71 | 20% Co/1.0% Re/0.5% P/Al2O3 | 78 | 0.89 | 190 | 17 |
| 72 | 20% Co/1% Re/1% P/Al2O3 | 43 | 0.91 | 380 | 8 |
| 73 | 20% Co/0.25% V/Al2O3 | 71 | 0.87 | 220 | 15 |
| 74 | 20% Co/1.0% Re/Al2O3(F) | 89 | 0.87 | 220 | 14 |
| 75 | 20% Co/1.0% Re/0.01% B/Al2O3(F) | 85 | 0.85 | 210 | 18 |
| 76 | 20% Co/1.0% Re/0.05% B/Al2O3(F) | 66 | 0.87 | 170 | 18 |
| 77 | 20% Co/1.0 Re/0.1% B/Al2O3(F) | 85 | 0.88 | 240 | 14 |
| 78 | 20% Co/1.0% Re/0.05% Li/Al2O3 | 60 | 0.89 | 220 | 24 |
| 79 | 20% Co/1.0% Re/0.1% Li/Al2O3 | 77 | 0.87 | 110 | 28 |
| 80 | 20% Co/1.5% Re/0.05% K/Al2O3 | 50 | 0.90 | 290 | 24 |

While preferred embodiments of this invention have been shown and described, modifications thereof can be made by one skilled in the art without departing from the spirit or teaching of this invention. The embodiments described herein are exemplary only and are not limiting. Many variations and modifications of the embodiments are possible and are within the scope of the invention. For example, the while the synthesis reaction of the some examples has been carried out in batch mode and of other examples has been carried out in continuous mode, it will be understood that the embodiments as described herein are not limited to the mode of reaction. Further, the steps of a process may be carried out in any suitable sequence. Accordingly, the scope of protection is not limited to the embodiments described herein, but is only limited by the claims that follow, the scope of which shall include all equivalents of the subject matter of the claims.

What is claimed is:

1. A catalyst comprising:
alumina;
cobalt;
rhenium; and
a promoter selected from the group consisting of boron, phosphorous, vanadium, manganese, and alkali metals;
wherein said catalyst is effective for producing hydrocarbons from synthesis gas; and
wherein said catalyst comprises said promoter in an amount effective for producing hydrocarbons from synthesis gas with an improved activity as compared to a similar catalyst without said promoter.

2. The catalyst according to claim 1 wherein said activity is improved by least about 5%.

3. The catalyst according to claim 1 wherein said hydrocarbons are characterized by a weight range suitable for processing to diesel fuel.

4. The catalyst according to claim 3 wherein said hydrocarbons comprise C₁₁₊ hydrocarbons.

5. The catalyst according to claim 3 wherein said hydrocarbons are characterized by a weight range suitable for processing to gasoline.

6. The catalyst according to claim 3 wherein said hydrocarbons comprise C₅₊ hydrocarbons.

7. The catalyst according to claim 1 wherein the weight ratio of said promoter to said rhenium is from about 0.01 to 1 to about 10 to 1 on a dry basis.

8. The catalyst according to claim 1 wherein said promoter is boron.

9. The catalyst according to claim 1 wherein said promoter is phosphorous.

10. The catalyst according to claim 1 wherein said promoter is manganese.

11. The catalyst according to claim 1 wherein said promoter is vanadium.

12. The catalyst according to claim 1 wherein said promoter is an alkali metal.

13. The catalyst according to claim 1 wherein the weight ratio of said rhenium to said cobalt is up to about 0.05 to 1 on a dry basis.

14. A process for producing hydrocarbons, comprising:
contacting a feed stream comprising carbon monoxide and hydrogen with a catalyst in a reaction zone maintained at conversion-promoting conditions effective to produce an effluent stream comprising hydrocarbons;
wherein the catalyst comprises alumina, cobalt, rhenium, and a promoter selected from the group consisting of boron, manganese, phosphorous, vanadium, and alkali metals; and
and wherein the presence of the promoter improves the catalyst activity.

15. The process according to claim 14 wherein the presence of the promoter increases a hydrocarbon productivity.

16. The process according to claim 15 wherein the productivity is increased by least about 5%.

17. The process according to claim 15 wherein said hydrocarbons are characterized by a weight range suitable for processing to diesel fuel.

18. The process according to claim 17 wherein said hydrocarbons comprise C₁₁₊ hydrocarbons.

19. The process according to claim 15 wherein said hydrocarbons are characterized by a weight range suitable for processing to gasoline.

20. The process according to claim 19 wherein said hydrocarbons comprise C₅₊ hydrocarbons.

21. The process according to claim 13 wherein the weight ratio of the promoter to the rhenium is from about 0.01 to 1 to about 10 to 1 on a dry basis.

22. The catalyst according to claim 13 wherein the weight ratio of said rhenium to said cobalt is up to about 0.05 to 1 on a dry basis.

23. The process according to claim 13 wherein the promoter is boron.

24. The process according to claim 13 wherein the promoter is phosphorous.

25. The process according to claim 13 wherein the promoter is manganese.

26. The process according to claim 13 wherein the promoter is vanadium.

27. A process for producing hydrocarbons, comprising:
contacting a feed stream comprising carbon monoxide and hydrogen with a catalyst in a reaction zone maintained at conversion-promoting conditions effective to produce an effluent stream comprising hydrocarbons;
wherein the catalyst comprises alumina, cobalt, rhenium, and a promoter selected from the group consisting of boron, manganese, phosphorous, vanadium, and alkali metals; and
wherein the catalyst is made by a process comprising:
adding a promoter to the catalyst in an amount effective to improve the activity of the catalyst by at least about 5%.

28. The process according to claim 27 wherein the adding comprises:
co-dispersing the promoter with the cobalt on a support.

29. The process according to claim 27 wherein the adding comprises:
layering the promoter over the cobalt.

30. The process according to claim 27 wherein the promoter is boron.

31. The process according to claim 27 wherein the promoter is manganese.

32. The process according to claim 27 wherein the promoter is phosphorous.

33. The process according to claim 27 wherein the promoter is vanadium.

34. The process according to claim 27 wherein the promoter is an alkali metal.

35. The process according to claim 34 wherein the promoter is selected from the group consisting of lithium and potassium.
